(19) United States
(12) Patent Application Publication
(10) Pub. No.: US 2023/0225656 A1
(43) Pub. Date: Jul. 20, 2023

(54) TRENCH ELECTRODE STRUCTURE FOR BIOSIGNAL MEASUREMENT

(71) Applicant: Murata Manufacturing Co., Ltd., Nagaokakyo-shi (JP)

(72) Inventor: Koji YOSHIDA, Smyrna, GA (US)

(21) Appl. No.: 18/181,033

(22) Filed: Mar. 9, 2023

Related U.S. Application Data
(63) Continuation of application No. PCT/US2021/050455, filed on Sep. 15, 2021.
(60) Provisional application No. 63/078,451, filed on Sep. 15, 2020.

(51) Int. Cl. A61B 5/259; A61B 5/266

(52) U.S. Cl. CPC A61B 5/259; A61B 5/266

(57) ABSTRACT

A conductive electrode that includes a substrate having first and second opposed main surfaces and a plurality of trenches extending between the first and second opposed main surfaces; and a conductive material within the plurality of trenches and extending between the first and second opposed main surfaces so as to provide an electrically conductive path between the first and second opposed main surfaces.

### CROSS-REFERENCE TO RELATED APPLICATIONS

This The present application is a continuation of International application No. PCT/US2021/050455, filed Sep. 15, 2021, which claims priority to U.S. Provisional Patent Application No. 63/078,451, entitled TRENCH ELECTRODE STRUCTURE FOR BIOSIGNAL MEASUREMENT, filed on Sep. 15, 2020, the entire contents of each of which are incorporated by reference herein in their entirety.

### FIELD OF THE INVENTION

The present invention relates to a conductive electrode with a low impedance and an increased contact area for better signal transmission. More particularly, the present invention relates to a dry conductive electrode with a low impedance and an increased contact area for better signal transmission.

### BACKGROUND OF THE INVENTION

Surface electrodes are types of electrodes applied to the skin of a subject for use in recording and evaluating the electrical activities of the heart (electrocardiography, i.e., ECG), skeletal muscles (electromyography, i.e., EMG) and neurons of the brain (electroencephalography, i.e., EEG) from the surface of the skin. The purpose of such a surface electrode is to act as a connector between a person's skin (where electrical signals are easiest to detect) and a detection unit via a lead cable.

There are two general types of surface electrodes, dry and wet. Dry electrodes are a type of surface electrode that do not use an aqueous electrolyte. One type of dry electrode has a simple structure that includes a metal plate connected with a lead wire. Other types of dry electrodes have a silicone elastomer base material with a conductive powder contained therein, and an electrode connector on the silicone elastomer for connection to the lead cable. These dry electrodes also typically include an adhesive on one side for adhering to the skin.

If a dry electrode makes good contact with the skin, the signals that are generated will be relatively accurate. Thus, one of the most important goals for any such dry electrode is to obtain a low contact impedance in order to attain a high signal-to-noise ratio. However, if an electrode does not adhere well to the skin or does not have a sufficient contact area with the skin, the resulting signal may contain an undue amount of noise which will interfere with accurate measurement.

Due to the deficiencies in present dry electrode designs, one known way to improve the contact impedance is to abrade the skin of the person so as to remove a layer of dead skin from the surface. This technique, however, can cause irritation to the skin of the person and is therefore not desirable.

Wet electrodes are typically constructed to have one or more pins that extend outward from the bottom of the electrode. These electrodes are then attached to the skin of a person with an elastic band after an aqueous electrode gel is placed between the electrode and the skin of the person. Such a system is uses additional materials and steps, can be messy with the use of the aqueous electrode gel, and more importantly, typically causes irritation to the skin of the person by the pins pressing into the skin.

### SUMMARY OF THE INVENTION

Thus, an object of the present invention is to provide a conductive electrode with a low impedance and an increased contact area for better signal transmission, and more particularly a dry electrode that has a low impedance and does not need an aqueous electrode gel in order to have a low signal-to-noise ratio.

This summary is provided to introduce a selection of concepts in a simplified form that are further described below in the DETAILED DESCRIPTION. This summary is not intended to identify key features of the claimed subject matter, nor is it intended to be used as an aid in determining the scope of the claimed subject matter.

According to an aspect of the present invention, the conductive electrode includes a substrate having first and second opposed main surfaces and a plurality of trenches extending between the first and second opposed main surfaces; and a conductive material within the plurality of trenches and extending between the first and second opposed main surfaces so as to provide an electrically conductive path between the first and second opposed main surfaces.

In a preferred embodiment, the conductive electrode further includes a lead-out electrode electrically connected to the conductive material in the trenches, and the lead-out electrode is on the first main surface of the substrate.

In a further preferred embodiment, the conductive material is MXene and the substrate is an adhesive conductive solid gel material.

In an another aspect of the present invention, the porous substrate has containing the plurality of trenches and a peripheral portion surrounding the central portion that does not contain the plurality of trenches. Most preferably, the central portion has a larger thickness than the peripheral portion such that a gap is formed between the central portion and the peripheral portion at the second main surface of the substrate.

Additional advantages and novel features of the present invention will be set forth in part in the description that follows, and in part will become more apparent to those skilled in the art upon examination of the following or upon learning by practice of the disclosure.

### BRIEF DESCRIPTION OF THE DRAWINGS

The novel features believed to be characteristic of the disclosure are set forth in the appended claims. In the description that follows, like parts are marked throughout the specification and drawings with the same numerals, respectively. The drawing figures are not necessarily drawn to scale and certain figures may be shown in exaggerated or generalized form in the interest of clarity and conciseness. The disclosure itself, however, as well as a preferred mode of use, further objects and advances thereof, will be best understood by reference to the following detailed description of illustrative aspects of the disclosure when read in conjunction with the accompanying drawings, wherein:

FIG. 1 is a top plan view showing the conductive electrode of a first embodiment of the present invention;

FIG. 2 is a cross section along line A-A of FIG. 1; and

FIG. 3 is a cross section showing a conductive electrode of a second embodiment of the present invention.

### DETAILED DESCRIPTION OF THE PREFERRED EMBODIMENTS

The detailed description set forth below in connection with the appended drawings is intended as a description of various configurations and is not intended to represent the only configurations in which the concepts described herein may be practiced. The detailed description includes specific details for the purpose of providing a thorough understanding of such various concepts. However, it will be apparent to those skilled in the art that these concepts may be practiced without these specific details. In some instances, well known structures and components are shown in block diagram form in order to avoid obscuring such concepts. Each embodiment is exemplified below, and it will be apparent that the structures shown in different embodiments can be partly replaced or combined with each other. In the second and subsequent embodiments, description of matters common to the first embodiment will be eliminated, and only different points will be described. In particular, a similar effect by a similar structure will not be sequentially referred to for each embodiment.

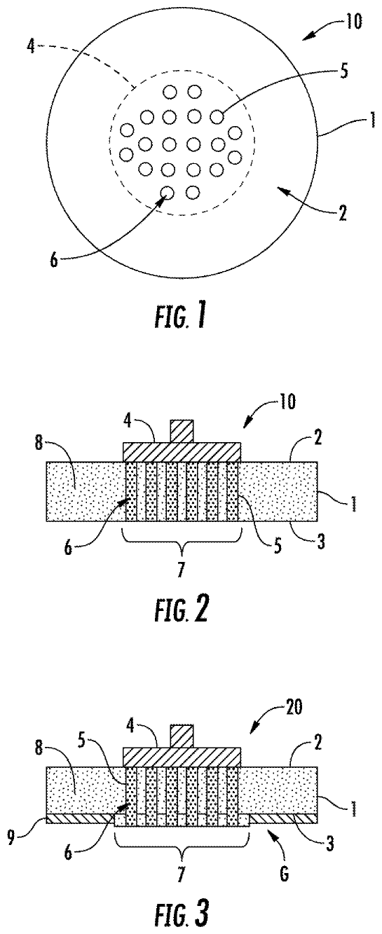

Referring now to FIGS. 1 and 2, a first embodiment of the present invention will be described. FIG. 1 is a top plan view showing the conductive electrode of the first embodiment, and FIG. 2 is a cross section along line A-A of FIG. 1.

As shown in FIGS. 1 and 2, the conductive electrode 10 includes a substrate 1 having first and second opposed main surfaces 2, 3, and a lead-out electrode 4 on the first main surface 2 of the substrate 1. The preferred material for the substrate 1 is an adhesive conductive solid gel material. Such materials are known and typically comprise a polymer matrix that has suspended therein an electrolyte and a solvent. An example of such a material is available from Sekisui Kasei under the tradename ST-gel. The substrate can also be made of a non-conductive solid gel polymer material or a polymer material, such as, for example, PAN, PMMA, PVDF, POE, PTFE, PE, PP, or nylon. Preferably, the substrate 1 has a thickness of 0.1 mm to 2.0 mm. The thickness of the substrate, however, is not critical and one skilled in the art will appreciate that the thickness of the substrate will depend on the nature of use of the conductive electrode. For example, when used for skin contact, the thickness will be set such that the substrate has sufficient flexibility to follow the contours of the surface of the skin. In addition, while the drawings illustrate the substrate and the lead-out electrode being circular, the shape of these components can take many other forms, such as square, rectangular, triangular, rhomboid, etc., without departing from the scope of the present invention.

The substrate includes a plurality of trenches 5 that extend between the first and second opposed main surfaces 2, 3. The trenches 5 preferably each have a diameter of 0.5 to 5.0 mm, and most preferably more than 1 mm. The trenches 5 are filled with a conductive material 6 that extends between the first and second opposed main surfaces 2, 3 and provides an electrically conductive path between the first and second opposed main surfaces 2, 3. If the material of the substrate is the preferred adhesive conductive solid gel material, then the trenches 5 filled with the conductive material 6 provide a further electrically conductive path between the first and second opposed main surfaces 2, 3. Such an arrangement will further reduce the impedance of the electrode. As shown in FIGS. 1 and 2, the substrate 1 preferably has a central portion 7 that includes the trenches 5, and a peripheral portion 8 that does not contain the trenches 5 and which surrounds the central portion 7. Preferably, an area occupied by the plurality of trenches 5 is less than 50% of an entire area of the first or the second main surfaces of the substrate. This can be seen in the top view of the conductive electrode 10 in FIG. 1, where the lead-out electrode 4 is in dashed lines for purposes of clarity. Further, and although not shown in the drawings, the plurality trenches 5 can be dispersed throughout the entirety of the substrate 1.

The lead-out electrode 4 on the first main surface 2 of the substrate 1 is electrically connected to the conductive material 6 within the plurality of trenches 5. The lead-out electrode 4 is provided for connection to a lead cable (not shown) which is then connected to a detection unit. However, since the first and second opposed main surfaces 2, 3 of the substrate 1 are electrically connected via the conductive material 6 in the trenches 5, the use of a lead-out electrode is optional and the lead cable can be directly secured to the first main surface 2 of the substrate 1. When a lead-out electrode is provided, it is preferred that the lead-out electrode be made from Ag, Ag/AgCl, Cu, Au, or C. The lead-out electrode can be formed by a printing process such as screen printing, flexographic printing, gravure printing, and the like. The lead-out electrode 4 can also be partly within and covered by the substrate 1 (not shown). Further, and depending on the material of construction, the lead-out electrode can also be stamped from a conductive sheet material and electrically connected to the conductive material 6 in the trenches 5 of the substrate 1 with a conductive glue or epoxy, for example.

The conductive material 6 preferably comprises at least one of Ag, Au, Cu, Al, Be, Mg, Ca, Na, Rh, Jr, carbon, carbon nanotubes, and graphene. While the particle size of the conductive material is not particularly limited, the conductive material preferably has an average particle size set such that the conductive material can be adequately filled within the trenches of the substrate so that electrical conductivity is established between the first and second main surfaces of the substrate. Preferably, the conductive material has a D50 cumulative particle size distribution from 100 μm to 100 nm. Depending on the material of the conductive material, the particles do not necessarily have to physically touch each other when extending between the first and second main surfaces within the trenches. All that is required is that the material, size, and arrangement of the conductive material within the trenches of the substrate be such that an electrical signal can be propagated between the first and second main surfaces of the substrate.

With this in mind, a most preferred material for the conductive material is MXene. MXene is particularly preferred because it is a two-dimensional material having a low dielectric constant, and can be easily placed within the trenches of the substrate. MXene is also particularly preferred because its two-dimensional arrangement can maintain conductivity even when the substrate containing the MXene within the trenches is stretched or bent. This is particularly useful in skin electrodes that need to conform to the different topologies of the skin of different people. The MXene particles preferably have a D50 cumulative particle size distribution of 20 μm to 500 μm, more preferably from 30 μm to 300 μm, as measured with an LA960 Laser Scattering Particle Size Distribution Analyzer available from Horiba, Ltd.

The conductive material 5 can be placed within the trenches of the substrate by various methods, including, but not limited to, creating a paste with the conductive material included therein and then using screen printing, inkjet printing, or the like, to place the conductive material within the trenches of the substrate. The particular method selected will depend on the diameter and depth of the trenches. For example, for trenches having a diameter of more than 1 mm, a screen printing method can be used where the squeeze angle is 60°, the ink application speed is 150 mm/s at a print head distance of 1.5 mm when using a conductive paste containing 30-35 wt % of a Ag solid conductive material, and having a viscosity of 350-400 Pa·s and a density of 1.6 g/cm³.

In addition, an adhesive material (not shown in FIG. 2) can be placed on the second main surface 3 of the substrate 1 so as to secure the electrode to the skin of a person.

With the structure described above with reference to FIGS. 1 and 2, a conductive electrode with a low impedance and an increased contact area for better signal transmission can be provided.

Referring now to FIG. 3, a second embodiment of the present invention will now be described. FIG. 3 is a cross section view showing a conductive electrode of the second embodiment.

As shown in FIG. 3, the conductive electrode 20 includes a substrate 1 having first and second opposed main surfaces 2, 3, and a lead-out electrode 4 on the first main surface 2 of the porous substrate 1 similar to the first embodiment of FIGS. 1 and 2. The substrate includes a plurality of trenches 5 that extend between the first and second opposed main surfaces 2, 3. The trenches 5 are filled with a conductive material 6 that extends between the first and second opposed main surfaces 2, 3 and provides an electrically conductive path between the first and second opposed main surfaces 2, 3.

As shown in FIG. 3, the substrate 1 preferably has a central portion 7 that includes the trenches 5 containing the conductive material 6, and a peripheral portion 8 that does not contain the trenches 5 containing the conductive material 6 and which surrounds the central portion 7. The lead-out electrode 4 on the first main surface 2 of the substrate 1 is electrically connected to the conductive material 6 in the trenches 5. Details of the materials, dimensions, etc., for these portions of the conductive electrode 20 are omitted from the description of the second embodiment as they are the same as those of the first embodiment.

Different form the first embodiment, the central portion 7 having the trenches 5 containing the conductive material 6 has a larger thickness than the peripheral portion 8. This structure creates a gap G between the second main surface 3 at the central portion 7 and the second main surface 3 at the peripheral portion 8. Preferably, the gap G is 0.1 mm to 5.0 mm. The structure shown in FIG. 3 is particularly useful when the material of the substrate 1 does not have separate conductivity outside of the trenches 5 filled with the conductive material 6. The increased thickness of the central portion 7 assists in maintaining contact of the conductive material 6 in the trenches 5 with the skin of the person, thereby assisting in reducing the impedance of the conductive electrode.

As shown in FIG. 3, an adhesive material 9 is preferably placed on the second main surface 3 of the substrate 1 at the peripheral portion 8. The adhesive material 9 can cover the entirety of the second main surface 3 along the peripheral portion 8, or can cover only a portion thereof. The adhesive material 9 us used to secure the conductive electrode to the skin of a person. Further, the providing of the gap G along with the adhesive 9 assists in ensuring that the substantial entirety of the surface area of the central portion 6 having the trenches 5 containing the conductive material 6 is in contact with the skin, thereby assisting in further reducing the impedance of the conductive electrode 20.

With the structure described above with reference to FIG. 3, a conductive electrode with a lower impedance and an increased contact area for better signal transmission can be provided.

While the invention herein has been described in connection with a conductive electrode for biosignal measurement, the present invention is also equally useable in other types of sensors such as, for example, gas sensors, ion sensors, etc. Thus, while the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

What is claimed is:

1. A conductive electrode comprising:
a substrate having first and second opposed main surfaces and a plurality of trenches extending between the first and second opposed main surfaces; and
a conductive material within the plurality of trenches and extending between the first and second opposed main surfaces so as to provide an electrically conductive path between the first and second opposed main surfaces.

2. The conductive electrode of claim 1, wherein the conductive material is MXene.

3. The conductive electrode of claim 2, wherein the MXene has a D50 cumulative particle size distribution of 20 μm to 500 μm.

4. The conductive electrode of claim 1, wherein the conductive material comprises at least one of Ag, Au, Cu, Al, Be, Mg, Ca, Na, Rh, Jr, carbon, carbon nanotubes, and graphene.

5. The conductive electrode of claim 1, wherein the substrate is an adhesive conductive solid gel material.

6. The conductive electrode of claim 1, wherein the substrate is a non-conductive solid gel polymer material.

7. The conductive electrode of claim 1, wherein the substrate comprises PAN, PMMA, PVDF, POE, PTFE, PE, PP, or nylon.

8. The conductive electrode of claim 1, wherein the plurality of trenches each have a diameter of 0.5 to 5.0 mm.

9. The conductive electrode of claim 1, further comprising an adhesive material on at least part of the second main surface of the substrate.

10. The conductive electrode of claim 1, wherein the substrate has a central portion containing the plurality of trenches and a peripheral portion surrounding the central portion that does not contain the plurality of trenches.

11. The conductive electrode of claim 10, wherein the central portion has a larger thickness than the peripheral portion such that a gap is formed between the central portion and the peripheral portion at the second main surface of the substrate.

12. The conductive electrode of claim 1, wherein an area of the plurality of trenches is less than 50% of an entire area of the first or the second main surfaces of the substrate.

13. The conductive electrode of claim 1, further comprising a lead-out electrode on the first main surface of the substrate and electrically connected to the conductive material in the plurality of trenches.

14. The conductive electrode of claim 13, wherein the conductive material is MXene.

15. The conductive electrode of claim 14, wherein the MXene has a D50 cumulative particle size distribution of 20 μm to 500 μm.

16. The conductive electrode of claim 13, wherein the conductive material comprises at least one of Ag, Au, Cu, Al, Be, Mg, Ca, Na, Rh, Ir, carbon, carbon nanotubes, and graphene.

17. The conductive electrode of claim 13, wherein the substrate is an adhesive conductive solid gel material.

18. The conductive electrode of claim 13, wherein the substrate is a non-conductive solid gel polymer material.

19. The conductive electrode of claim 13, wherein the substrate comprises PAN, PMMA, PVDF, POE, PTFE, PE, PP, or nylon.

20. The conductive electrode of claim 13, wherein the plurality of trenches each have a diameter of 0.5 to 5.0 mm.

21. The conductive electrode of claim 13, further comprising an adhesive material on at least part of the second main surface of the substrate.

22. The conductive electrode of claim 13, wherein the substrate has a central portion containing the plurality of trenches and a peripheral portion surrounding the central portion that does not contain the plurality of trenches.

23. The conductive electrode of claim 22, wherein the central portion has a larger thickness than the peripheral portion such that a gap is formed between the central portion and the peripheral portion at the second main surface of the substrate.

24. The conductive electrode of claim 13, wherein an area of the plurality of trenches is less than 50% of an entire area of the first or the second main surfaces of the substrate.
